(19) United States
(12) Patent Application Publication
(10) Pub. No.: US 2014/0135567 A1
(43) Pub. Date: May 15, 2014

(54) Dual-Balloon Cardiac Pump

(71) Applicant: Charles Marotta, Chester, NJ (US)

(72) Inventor: Charles Marotta, Chester, NJ (US)

(21) Appl. No.: 13/674,496

(22) Filed: Nov. 12, 2012

(51) Int. Cl. A61M 1/10; A61M 1/12

(52) U.S. Cl. CPC A61M 1/1072; A61M 1/1086; A61M 1/12; A61M 1/106
USPC 600/17

(57) ABSTRACT

A dual-balloon cardiac pump comprises a flexible catheter tube in pneumatic communication with inflatable ventricular and aortic balloons, which are inflated and deflated in accordance with a programmed sequence of pressurized inert gas into the catheter tube as regulated by a control unit. The programmed sequence rapidly inflates and deflates the ventricular balloon after left ventricular contraction is completed. The programmed sequence inflates the aortic balloon after the aortic valve closes and deflates it just before the next systolic cycle begins.

### BACKGROUND OF THE INVENTION

The present invention relates to the field of heart assisting devices and more particularly to heart assisting devices that utilize one or more balloon pumps in the ventricles and/or aorta to achieve complete ejection of blood during systole.

Degeneration of the left ventricle in certain pathological heart conditions can cause diminished contraction and incomplete emptying of the heart during systole. This results in a “dead volume” of blood that remains in the ventricle at the end of systole and can lead to congestive heart failure.

The prior art cardiac assist balloon pumps include double balloon catheters for pumping blood from the left ventricle to the aorta synchronously with the left ventricle systolic and diastolic cycles. Examples of such devices are disclosed in the U.S. patents of Chey et al. (U.S. Pat. No. 4,902,273) and Segalowitz (U.S. Pat. No. 5,176,619). In these designs, the ventricular balloon is inflated during systole and deflated during diastole, while the aortic balloon is conversely inflated during diastole and deflated during systole. Hence, while the ventricular balloon is inflating, the aortic balloon is deflating, and vice-versa.

The disadvantage of the prior art design lies in the fact that the outward pushing force exerted by the inflating ventricular balloon is in opposition to the inward contracting force of the ventricle muscle, which tends to diminish the overall ejection efficiency.

In the present invention, on the other hand, the inflation of the ventricular balloon is deferred until the end of ventricular contraction, just before the isovolumic relaxation period. The ventricular balloon is rapidly inflated and deflated before the aortic valve closes, so that the residual blood remaining in the left ventricle is pushed out into the aorta. After the aortic valve closes, the aortic balloon inflates to push the blood through the aorta, and it deflates just before the heart contracts again to initiate the next cycle.

In this way, the pressure boost provided by the ventricular balloon is introduced after the ventricle contracts so as to supplement the force of the contraction rather than impede it, as the prior art devices do.

### SUMMARY OF THE INVENTION

The present invention is a dual-balloon cardiac pump comprising a flexible catheter tube in pneumatic communication with an inflatable distal ventricular balloon and an inflatable proximal aortic balloon. The dual balloon cardiac pump is introduced percutaneously through a peripheral artery and inserted into the left ventricle and the aorta.

The shape of the inflated ventricular balloon is spherical, ellipsoidal or frusto-conical, and it is sized to fit within the left ventricle. The shape of the inflated aortic balloon is oblong cylindro-spherical or cylindro-ellipsoidal, and it is sized to fit within the aorta. The distal tip of the catheter tube has a sensor for detecting pressure and/or electrical activity in order to synchronize inflation and deflation of the ventricular and aortic balloons.

The dual-balloon cardiac pump further comprises a control unit that supplies pressurized inert gas into the catheter tube so as to inflate and deflate the ventricular and aortic balloons in accordance with a programmed sequence. The programmed sequence rapidly inflates and deflates the ventricular balloon after left ventricular contraction is completed. The programmed sequence inflates the aortic balloon after the aortic valve closes and deflates the aortic balloon just before the next systolic cycle begins.

The foregoing summarizes the general design features of the present invention. In the following sections, specific embodiments of the present invention will be described in some detail. These specific embodiments are intended to demonstrate the feasibility of implementing the present invention in accordance with the general design features discussed above. Therefore, the detailed descriptions of these embodiments are offered for illustrative and exemplary purposes only, and they are not intended to limit the scope either of the foregoing summary description or of the claims which follow.

### BRIEF DESCRIPTION OF THE DRAWINGS

FIG. 1 is a perspective view of the preferred embodiment of the present invention;

FIG. 2 is a cutaway view of the preferred embodiment of the present invention inserted into a heart;

FIG. 3 is a schematic view of the programmed sequence of the preferred embodiment of the present invention during diastole;

FIG. 4 is a schematic view of the programmed sequence of the preferred embodiment of the present invention during early systole;

FIG. 5 is a schematic view of the programmed sequence of the preferred embodiment of the present invention during the isovolumic relaxation period in later systole.

### DETAILED DESCRIPTION OF THE PREFERRED EMBODIMENT

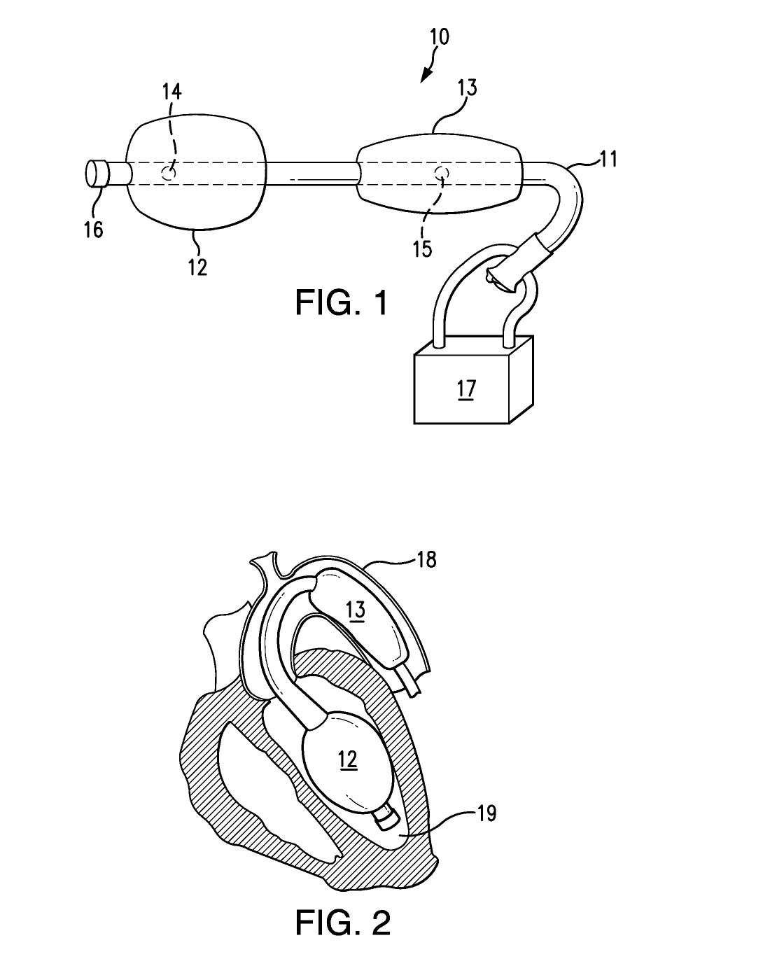

Referring to FIGS. 1 and 2, the preferred embodiment of the dual-balloon cardiac pump 10 comprises a flexible catheter tube 11 pneumatically serially connected to an inflatable distal ventricular balloon 12 and an inflatable proximal aortic balloon 13. The ventricular balloon 12 is preferably ellipsoidal and fits within the left ventricle 19 of the heart. The aortic balloon 13 is preferably oblong cylindro-ellipsoidal and fits within the aorta 18.

The ventricular balloon 12 is inflatable through a first aperture 14 (or a plurality of such apertures) in the catheter tube 11, and the aortic balloon 13 is inflatable through a second aperture 15 (or a plurality of such apertures) in the catheter tube 11. A sensor 16 in the distal tip of the catheter tube 11 detects pressure and/or electrical activity to synchronize inflation and deflation of the ventricular 12 and aortic 13 balloons.

A control unit supplies pressurized inert gas to the catheter tube 11 so as to inflate the ventricular 12 and aortic 13 balloons in accordance with a programmed sequence.

Referring to FIG. 2, the dual-balloon cardiac pump 10 is inserted into the heart so that the catheter tube 11 extends through the aorta 18 into the left ventricle 19. The ventricular balloon 12 is positioned in the left ventricle 19 and the aortic balloon 13 in the aorta 18.

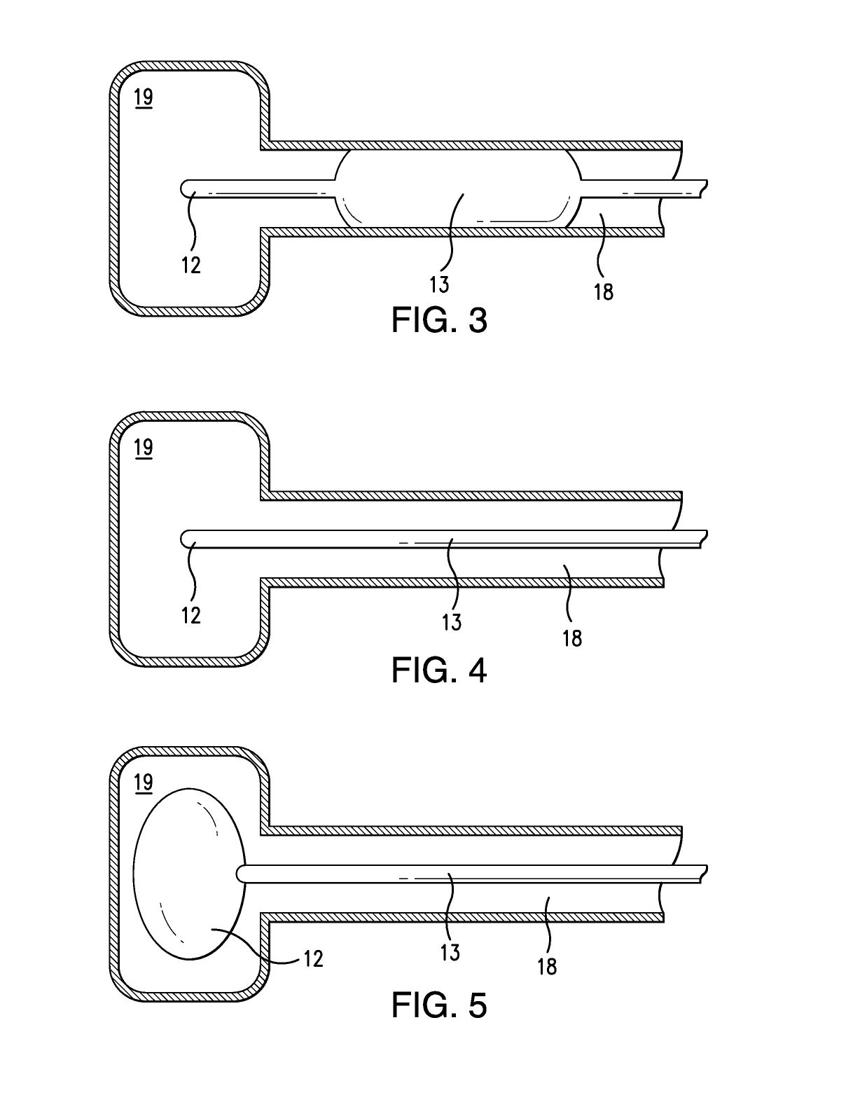

Referring to FIGS. 3-5, the programmed sequence consists of three stages. As shown in FIG. 3, during diastole, just after the aortic valve closes, the aortic balloon 13 is inflated and the ventricular balloon 12 remains deflated. As shown in FIG. 4, during early systole, both the aortic 13 and the ventricular 12 balloons are deflated. As shown in FIG. 5, later, at the end of ventricular contraction, just before the isovolumic relaxation period, the ventricular balloon 12 is rapidly inflated and deflated, while the aortic balloon 13 remains deflated.

Although the preferred embodiment of the present invention has been disclosed for illustrative purposes, those skilled in the art will appreciate that many additions, modifications and substitutions are possible, without departing from the scope and spirit of the present invention as defined by the accompanying claims.

What is claimed is:

1. A dual-balloon cardiac pump, inserted into a left ventricle and an aorta of a patient's heart, comprising:
a flexible catheter tube in pneumatic communication with an inflatable distal ventricular balloon and an inflatable proximal aortic balloon;
wherein the ventricular balloon is shaped and sized to fit within the left ventricle and is inserted into the left ventricle;
wherein the aortic balloon is shaped and sized to fit within the aorta and is inserted into the aorta;
wherein the catheter tube extends through the aorta into the left ventricle;
wherein the distal tip of the catheter tube has a sensor for detecting pressure or electrical activity, or both, in order to synchronize inflation and deflation of the ventricular and aortic balloons in a programmed sequence;
wherein the dual-balloon cardiac pump further comprises a control unit that supplies pressurized inert gas into the catheter tube so as to inflate and deflate the ventricular and aortic balloons in accordance with the programmed sequence; and
wherein the programmed sequence comprises three stages: (1) during diastole, immediately after the aortic valve closes, the aortic valve is inflated and the ventricular balloon remains deflated; (2) during early systole, the aortic balloon is deflated and the ventricular balloon remains deflated; and (3) after the contraction of the left ventricle is completed, just before the isovolometric relaxation period, the ventricular balloon is rapidly inflated and deflated, while the aortic balloon remains deflated.

2. The dual-balloon cardiac pump according to claim 1, wherein the ventricular balloon is inflatable through one or more apertures in the catheter tube, and the aortic balloon is inflatable through one or more apertures in the catheter tube.

3. The dual-balloon cardiac pump according to claim 2, wherein the shape of the ventricular balloon is ellipsoidal and the shape of the aortic balloon is oblong cylindro-ellipsoidal.
